(19) United States
(12) Patent Application Publication
(10) Pub. No.: US 2022/0002155 A1
(43) Pub. Date: Jan. 6, 2022

(54) Process for workup of mixed acid and wastewater from the nitration of aromatics and apparatus for performing the process

(71) Applicant: PLINKE GMBH, Bad Homburg (DE)

(72) Inventor: Hansjürgen WINTERBAUER, (US)

(21) Appl. No.: 17/286,450

(22) PCT Filed: Oct. 17, 2019
(86) PCT No.: PCT/EP2019/078207

§ 371 Date: Apr. 16, 2021

(30) Foreign Application Priority Data
Oct. 19, 2018 (DE) 10 2018 217 955.7

(51) Int. Cl. C01B 17/88; C01B 17/94

(52) U.S. Cl. CPC C01B 17/88; C01B 17/94

(57) ABSTRACT

In a process for workup of mixed acid and wastewater from the nitration of aromatics in which the nitric acid present is converted by reaction with an aromatic under adiabatic conditions,
a. at least one waste stream component selected from waste acid (mixed acid) generated in the nitration, acidic washing water from the workup of crude nitroaromatics and dilute nitric acid generated in an off-gas treatment in the course of the nitration is provided,
b. the at least one waste stream component is mixed with re-concentrated sulfuric acid,
c. an aromatic is added to the mixture in stoichiometric excess based on the nitric acid,
d. the obtained reaction mixture is reacted in an adiabatically operated reactor,
e. the obtained organic phase is separated from the sulfuric-acid-containing phase in a separator,
f. the sulfuric-acid-containing phase is concentrated under vacuum and
g. at least one substream of the re-concentrated sulfuric acid from step g) is employed in step b).

### FIELD OF THE INVENTION

The invention relates to a process for workup of waste stream components from the nitration of aromatics, in which the nitric acid present therein is reacted under adiabatic conditions by reaction with an aromatic. The invention further relates to an apparatus for performing the process.

### BACKGROUND OF THE INVENTION

In the production of nitroaromatic compounds by nitration using mixed acid, various waste streams are generated. These include the mixed acid used in the reaction, which is diluted during the reaction, acidic washing water from the workup of the crude nitroaromatics, and dilute nitric acid recovered during the off-gas treatment.

A process for purifying and concentrating used, contaminated sulfuric acids generated during the nitration of aromatic hydrocarbons in the presence of sulfuric acid is already known from DE 196 36 191 A1. Here, the steam-volatile compounds are fully removed by breaking down the nitrogen-containing compounds, and the sulfuric acid thus purified is concentrated. The contaminated sulfuric acid is preheated and freed of steam-volatile compounds in counterflow with the vapours of the first concentration step at pressures between 200 and 1000 mbar. The sulfuric acid is passed to a first concentration step, in which it is concentrated at the same pressure with an indirect supply of heat, and the sulfuric acid is subsequently concentrated to 88 to 97% by weight in a single- or multi-step vacuum concentration process at a pressure less than that in the first concentration step.

The crude nitroaromatic coming from the nitration is contaminated with residues of the nitric acid and with by-products, and has to be washed repeatedly. First, the acid residues are removed using an acid wash. The wastewater generated during the acid wash contains larger amounts of nitric acid and sulfuric acid, as is disclosed in EP 0 736 514 A1 using the example of dinitrotoluene.

DE 10 2006 013 579 B3 discloses a process for reducing the wastewater amount in the production of dinitrotoluene (DNT) and simultaneously optimising the wastewater quality by reducing the proportion of organic impurities, in which, in a first step, after preheating to 150 to 170° C., the waste acid generated during the nitration is stripped under atmospheric pressure conditions in a column in counterflow with water vapour which is produced by concentrating the sulfuric acid running out of the column. A strip vapour amount of between 0.25 and 10% by weight based on the waste acid amount is used, and a nitric acid comprising 20 to 40% by weight HNO₃ is thus obtained at the head of the column. The nitric acid is subsequently fed back into the nitration process, directly or after being concentrated. In a second step, in a vacuum of between 200 and 600 mbar, the pre-purified waste acid from the atmospheric stripping is stripped in a column in counterflow with water vapour which is produced by concentrating the sulfuric acid running out of the column, with a strip vapour amount of between 5 and 10% by weight based on the incoming pre-purified waste acid amount being used, and a condensate which can be used again in the acid wash of the DNT thus being obtained at the head of the column. In a downstream sulfuric acid concentration means, the sulfuric acid running out of the vacuum stripping is concentrated to a concentration of between 85 and 98% H₂SO₄ in one or more steps in a vacuum of between 150 and 30 mbar, preferably between 100 and 50 mbar, and a condensate is thus obtained which, as well as small amounts of sulfuric acid, still contains just traces of nitroaromatic compounds, at concentrations of <100 ppm. Nitrous off-gases from the individual process steps are purified by absorption of the NOₓ in counterflow with water, and nitric acid is thus recovered, which is subsequently fed back into the nitration process, directly or after being concentrated.

EP 2 295 375 B1 discloses a process for workup of waste acid from the production of nitroaromatics, in particular the production of dinitrotoluene (DNT) or trinitrotoluene (TNT), to obtain concentrated and purified sulfuric acid and nitric acid, in which, in a first step, the preheated waste acid, which as well as up to 80% by mass sulfuric acid and water contains nitric acid (HNO₃), nitrosylsulfuric acid (as HNO₂) and nitroorganics, in particular DNT and mononitrotoluene (MNT), as further components, is separated into at least one vapour phase, containing nitric acid with or with or without nitroorganics, and a pre-concentrated sulfuric acid in a strip column in counterflow with vapour which is obtained from the base of the strip column by heating the pre-concentrated sulfuric acid. In downstream process steps, (i) the pre-concentrated sulfuric acid obtained from the base of the strip column is supplied to further purification for removing nitroorganics and for higher concentrations, and (ii) the nitric acid obtained from the vapour nitric acid phase and the nitroorganics, including the nitroorganics obtained during the further purification and concentration of the pre-concentrated sulfuric acid, are worked up and supplied back into the nitration process. This process is further characterised in that, in the first process step, as well as the stripping of the preheated waste acid in a strip column in counterflow with vapour from the sulfuric acid enrichment (V1), the nitric acid present in the strip vapour is concentrated in counterflow with additional purified and optionally fresh concentrated sulfuric acid having a concentration in a range of 75 to 97% by mass and preferably 80 to 96% by mass. The nitric acid vapours obtained from the top of the column in the first step are condensed into a nitric acid directly in a highly concentrated form suitable for feeding back into the nitration process.

U.S. Pat. No. 4,496,782 A discloses processes for recovering nitric acid from the used acid phase of a mixed acid mononitration reaction, comprising adding a sufficient amount of nitric acid so as to provide at least approximately 2% by weight nitric acid concentration in the used acid phase from the mononitration reaction. By adiabatic reaction of a mononitroaromatic hydrocarbon in a more than stoichiometric amount with the nitric acid in the used acid phase, a dinitroaromatic hydrocarbon product and a nitric acid concentration of less than approximately 0.25% by weight in the used acid phase are subsequently obtained.

U.S. Pat. No. 4,650,912 A discloses a process for denitrification of the used acid phase containing sulfuric acid and nitric acid from the nitration of an aromatic hydrocarbon by the mixed acid process, comprising forming a denitrification medium by contacting the used acid phase with an aromatic hydrocarbon under nitration reaction conditions to obtain the nitric acid by forming a nitroaromatic hydrocarbon. An amount of aromatic hydrocarbon is added which is slightly less than or equal to the stoichiometric amount required to break down the used acid phase of the nitric acid, and the denitrification reaction medium is photometrically monitored for the appearance of a dark-red to black colour, whereupon, when a colour of this type is detected, the molar ratio of aromatic hydrocarbon to nitric acid in the denitrification reaction medium is adjusted so as to eliminate the colour.

U.S. Pat. No. 8,907,144 B2 discloses a process for continuous adiabatic nitration of toluene into mononitrotoluene (MNT). The process results in an MNT product quality comparable to that of isothermal production. The process uses excess toluene, the reaction rate being controlled in such a way that a residue of 0.003-0.102% by weight nitric acid in the used acid and an orange-red colour of the used acid are obtained. Further process conditions are re-concentrated sulfuric acid at 83 to 99° C. having a sulfuric acid concentration of 66 to 70.5% by weight. This is mixed with nitric acid to form a mixed acid comprising 1.0 to 3.8% by weight nitric acid, and toluene is added in an amount of 1.1 to 1.71 mol toluene per mol nitric acid.

Although the processes disclosed in U.S. Pat. Nos. 4,496,782 A and 4,650,912 A ought to be much more favourable than the process comprising stripping, since they react the nitric acid, which therefore does not have to be re-concentrated, they have not become widespread. Both processes only add the aromatic compound to the nitric acid in a sub-stoichiometric to maximum stoichiometric ratio, and so complete reaction of the nitric acid does not occur. In U.S. Pat. No. 4,650,912 A, the appearance of a dark red to black colouration is even described, which occurs in the event of an over-stoichiometric addition of the aromatics and impedes reliable continuous operation of this process. Both processes are also limited to workup of the mixed acid from the nitration reaction, and the other waste streams containing nitric acid have to be treated separately. In the process for adiabatic nitration disclosed in U.S. Pat. No. 8,907,144 B2, although toluene is added in stoichiometric excess, in this case too the reaction is controlled in such a way that not all of the nitric acid is reacted. Also, this is neither a process for adiabatic production of MNT nor a process for workup of the waste acid from the isothermal nitration of aromatics.

### Objective of the Invention

Now, the object of the process according to the invention is efficient workup of the waste streams containing nitric acid which are generated during nitration of aromatics. Preferably, all relevant components, namely the mixed acid used, which is diluted in the reaction, the acid washing water from the workup of the crude nitroaromatics, and the dilute nitric acid generated during the off-gas treatment, are to be worked up together.

### SUMMARY OF THE INVENTION

This object is achieved according to the invention by a process for workup of waste streams from the nitration of aromatics, in which nitric acid present therein is reacted under adiabatic conditions by reaction with an aromatic, characterised in that
a) at least one waste stream component selected from waste acid (mixed acid) generated in the nitration, acidic washing water from the workup of crude nitroaromatics and dilute nitric acid generated in an off-gas treatment in the course of the nitration is provided,
b) the at least one waste stream component is mixed with re-concentrated sulfuric acid,
c) an aromatic is added to the mixture in stoichiometric excess based on the nitric acid,
d) the obtained reaction mixture is reacted in an adiabatically operated reactor,
e) the obtained organic phase is separated from the sulfuric-acid-containing phase in a separator,
f) the sulfuric-acid-containing phase is concentrated under vacuum and
g) at least one substream of the re-concentrated sulfuric acid from step g) is employed in step b).

In a preferred embodiment of the invention, at least two waste stream components and particularly preferably all the above-mentioned waste stream components are pre-mixed in step a) and subsequently mixed with the re-concentrated sulfuric acid.

### BRIEF DESCRIPTION OF THE DRAWINGS

FIG. 1 Simplified block flow diagram of the workup of mixed acid and wastewater from the nitration of aromatics by the process according to the invention in the production of DNT

FIG. 2 Apparatus according to the invention for performing the process for workup of mixed acid and wastewater from the nitration of aromatics

### DETAILED DESCRIPTION OF THE INVENTION

The process according to the invention makes it possible to free the nitric-acid-containing waste streams from nitration processes completely of nitric acid, preferably jointly by adiabatic nitration and reaction with an aromatic or nitroaromatic, and to configure the reaction conditions and the reactor in such a way that the problems described in similar processes do not occur and the process also holds up in practical operation. This can surprisingly be achieved simply by using a continuously operating reactor and feeding back a substream of the re-concentrated, purified sulfuric acid. By proceeding in this manner, it is surprisingly possible to use the aromatic compound in stoichiometric excess based on the nitric acid present in the mixture, meaning that the nitric acid can be completely neutralised. The problems described in U.S. Pat. No. 4,650,912 A for over-stoichiometric addition are surprisingly not observed in the process according to the invention.

Hereinafter, the process according to the invention is described using the example of nitration of toluene into dinitrotoluene. All pressure specifications are given in the form of absolute pressure. The process is of course also applicable to all other aromatic compounds which can undergo single, double or even multiple nitration. Examples include nitration of benzene to nitrobenzene or dinitrobenzene, of toluene to mononitrotoluene (MNT), dinitrotoluene (DNT) or trinitrotoluene (TNT), of nitrochlorobenzene (NCB) to mononitrochlorobenzene (MNCB) or dinitrochlorobenzene (DNCB), etc. The specified aromatics are mentioned purely by way of example, and do not limit the scope of application of the process according to the invention, meaning that all specifications and preferred embodiments also apply to the nitration of other aromatics.

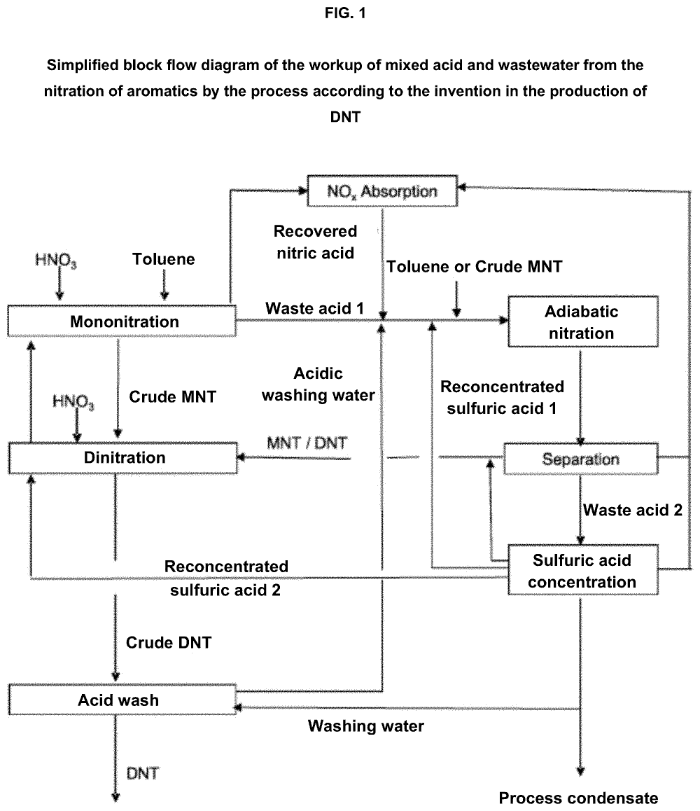

FIG. 1 shows a simplified block flow diagram for a process for producing DNT with acid workup using the process according to the invention. In the mononitration, toluene is nitrated into mononitrotoluene by adding nitric acid and sulfuric acid from the dinitration. The crude MNT obtained, a mixture of the various isomers along with a proportion of DNT, is subsequently further nitrated into DNT in dinitration with further addition of nitric acid and re-concentrated sulfuric acid. The crude dinitrotoluene thus obtained is largely freed of remaining nitric acid and sulfuric acid in an acid wash.

It is preferred according to the invention for the waste acid 1 generated from the mononitration to be mixed with the acid washing water from the acid wash and the recovered nitric acid from an NOₓ absorption conventionally included in nitrations. According to the invention, the mixing may take place in a simple container. Alternatively, however, prior art mixing apparatuses such as stirring apparatuses, static mixers or the like may also be used.

The mixture is preferably heated by recovering the energy of the re-concentrated sulfuric acid 2 obtained in the sulfuric acid concentration process, which acid is thus cooled. However, heating may also be provided by indirect steam heating or a combination of steam heating and energy recovery. The preheating of the mixture is adjusted in such a way that the temperature after the addition of the re-concentrated sulfuric acid 1 is in a range of 70° C. to 130° C. Subsequently, the uncooled re-concentrated sulfuric acid 1 is added. According to the invention, the re-concentrated sulfuric acid 1 fed to the adiabatic nitration may have a concentration the same as or lower than that of the re-concentrated sulfuric acid 2 used in the dinitration. According to the invention, in this context the amount and concentration of the re-concentrated sulfuric acid 1 is selected in a manner resulting in a mixed acid having a sulfuric acid content of between 60% and 70% H₂SO₄ and a nitric acid content of between 1% and 5% HNO₃. The required concentration and temperature in the concentration step can be established by way of the vacuum set during the concentration step. On this matter, a person skilled in the art may make use of the current literature on material values of sulfuric acid, such as Perry's Chemical Engineers' Handbook (McGraw Hill).

If re-concentrated sulfuric acid from a preceding process is not present in a start-up phase of the reaction, fresh sulfuric acid of a corresponding concentration may be used instead.

The temperature at the reactor input is set in a range of 70° C. to 130° C., preferably in a range of 90° C. to 110° C., in such a way that the reaction initialises directly after the addition of the aromatic compound. By way of a special adding system, the aromatic, for example toluene or MNT, for the nitration reaction is added to the circulating mixture of sulfuric acid, nitric acid and water, and finely distributed. As an alternative to pure MNT as an individual isomer or as an isomer mixture, crude MNT from the mono-nitration, containing a proportion of DNT, may also be used here. In the adiabatic nitration, in an adiabatically operated reactor, the nitric acid present in the mixture and comprising the aromatics is then virtually completely reacted to form the nitroaromatic, In the case of toluene, predominantly MNT is obtained here as an isomer mixture; if MNT is used, predominantly DNT is obtained in the reaction.

A tube reactor is preferably used as the reactor. Stirred reactors are also possible according to the invention. The tube reactor, however, has the advantage that it requires significantly less space. The tube reactor is preferably modular in construction, and contains static mixing elements for remixing the reaction media. According to the invention, the number of static mixing elements is between 2 and 20. The mixing elements result in a loss of pressure over the reactor. According to the invention, the operating pressure at the reactor input is in a range of 1 bar to 10 bar, preferably in a range of 3 bar to 6 bar.

The aromatic is added in a stoichiometric excess in a range of 1%-20%, preferably 3%-10%, based on the nitric acid amount, so as to ensure complete reaction. Greater excess would also be possible and would not interfere with the reaction, but is not desired because the organic substances are separated off again in the following step.

After the nitric acid is reacted in the adiabatic reactor, the sulfuric acid and the organic product phase are separated. This is preferably done by exploiting the different densities of the sulfuric acid and the obtained nitroaromatics, presently for example a mixture of toluene and MNT or of MNT and DNT. As separators, simple prior art containers, with or without installations, or also centrifuges may be used. According to the invention, the pressure in the separator is preferably between 1 bar and 2 bar. This prevents the product mixture of aromatic and/or nitroaromatic being partially evaporated and the evaporation impeding the separation. The pressure loss over the reactor corresponds to the inputted mixing energy. The separated-off organic phase can then be fed into the mononitration and/or dinitration for further nitration, separately or together with the organic compounds recovered during the sulfuric acid concentration process, or can alternatively be worked up directly.

Surprisingly, the colour appearances described in U.S. Pat. No. 4,650,912 A for over-stoichiometric addition of the aromatic are not observed in the process according to the invention. This could be due to the increased temperature and the different acid composition resulting from feeding back the purified re-concentrated sulfuric acid.

As a result of the adiabatic procedure, the separated waste sulfuric acid 2 has absorbed the reaction energy and thus according to the invention been heated by 10 to 40° C. The waste acid 2 is flashed in an evaporator, which is preferably operated at a pressure of between 30 mbar and 500 mbar, and thus concentrated. The sulfuric acid is further concentrated using the conventional prior art apparatuses for concentrating sulfuric acid, and this may take place in one or more steps, depending on the system performance. If necessary, organic solvent may also be added to the condensation system of the sulfuric acid concentration process, in accordance with the prior art, so as to prevent deposits of nitroaromatics having a higher melting point. The re-concentrated sulfuric acid 1 is fed back to the adiabatic nitration. The remaining sulfuric acid may optionally be further concentrated using the prior art processes, and is fed to the dinitration as re-concentrated sulfuric acid 2. The further concentration takes place in a vacuum of between 150 and 30 mbar, preferably between 100 and 50 mbar, to the desired final concentration of between 85 and 98% H₂SO₄. Depending on what concentration is used in the associated nitration process, any desired sulfuric acid concentration as required for the nitration process may be set for the re-concentrated sulfuric acid 2. The specified concentrations are selected purely by way of example, and are not intended to limit the process.

Part of the process condensate from the sulfuric acid concentration process may be used for the acid wash. All off-gases generated in the process steps are passed to the NOₓ absorption, and the nitrous gases present are recovered as nitric acid. According to the invention, the nitric acid thus obtained is mixed with the waste acid 1 and fed into the adiabatic nitration for the reaction.

The main advantage of the process according to the invention over the prior art is that all of the nitric acid is reacted and does not have to be re-concentrated. In the currently used processes according to DE 10 2006 103 579 B3 and EP 2 295 375 B1, in which the nitric acid is stripped out of the waste sulfuric acid, energy is required for this stripping and for the subsequent concentration of the nitric acid. In the process according to the invention, however, no energy supply is required; on the contrary, as a result of the adiabatic procedure, even the released reaction energy is still utilised for re-concentrating the sulfuric acid. Also, in the process according to the invention, no columns are required for the stripping, meaning that the building required for setting up the apparatus for performing the process can be built much lower, and the investment costs are thus reduced accordingly.

The present invention also relates to an apparatus for performing the process according to the invention.

The apparatus consists of a mixing unit in which the waste acid from the nitration, the recovered dilute nitric acid from the absorption of nitrous gases in the course of the process, and the acidic washing water from the acid wash of the nitroaromatics are mixed. The apparatus further comprises at least one heat exchanger for preheating the obtained mixture which is to be worked up by the process according to the invention, and a pump by means of which this mixture is mixed with sulfuric acid, in particular a re-concentrated sulfuric acid from the process according to the invention, and which can generate the pressure required at the reactor input. The apparatus further comprises an injection unit, in which the aromatic to be nitrated is added to the mixture via nozzles and finely distributed. The apparatus further comprises a modularly constructed tube reactor, which comprises between 2 and 20 static mixers and in which the aromatic is nitrated, and a separator which is downstream of the tube reactor and in which the mixture exiting the tube reactor is separated into an organic phase and an acid phase. The apparatus according to the invention further comprises a flash evaporator, a concentration unit comprising an indirectly heated heat exchanger and an associated evaporator having associated vapour condensation, and a vacuum unit.

In the context of the present invention, it is thus preferred for a single-step or multi-step sulfuric acid concentration means to be installed, in which residual sulfuric acid which is not fed to the adiabatic nitration is further concentrated.

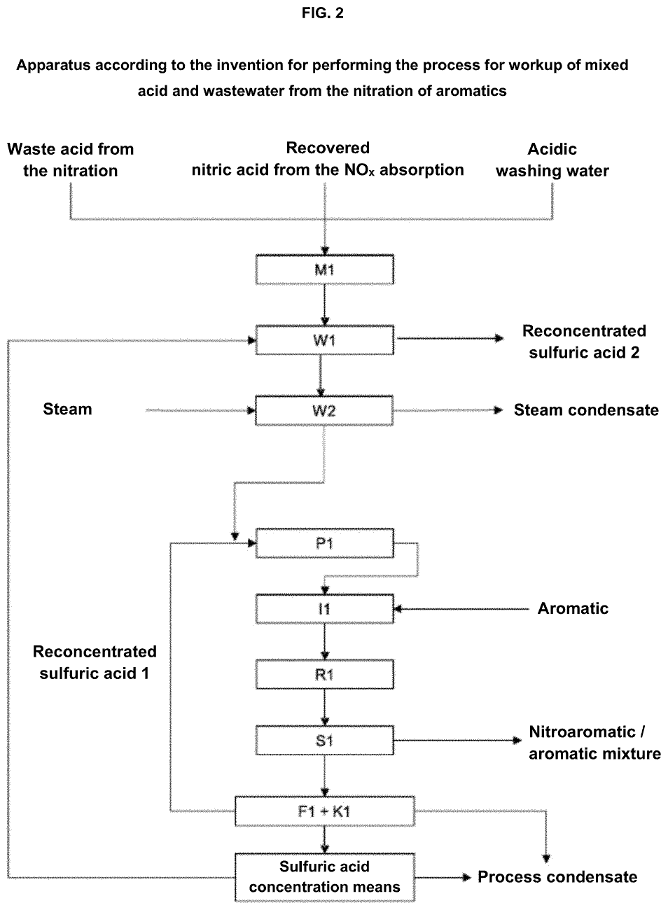

FIG. 2 shows the simplified construction of an apparatus according to the invention for performing the process according to the invention. The apparatus according to the invention consists of a mixing unit M1, in which the waste acid from the nitration, the recovered nitric acid from the NOₓ absorption, and the acidic washing water from the nitroaromatic wash are mixed. A simple tank without installations can serve as the mixing unit M1. Alternatively, a tank comprising a stirring apparatus or an apparatus comprising static mixers may also be used.

The mixture is subsequently preheated in a heat exchanger W1, preferably by indirect heat exchange with the re-concentrated sulfuric acid 2 running off from the sulfuric acid concentration means and with an indirectly steam-heated heat exchanger W2. Optionally, it is also possible for only one of the two preheaters to be used. The preheated mixture is added on the suction side of the pump P1, together with re-concentrated sulfuric acid from the flashing F1 and concentrating K1. The pump P1 mixes the two streams and generates the liquid pressure required at the reactor input. The mixture flows through the injection unit I1, in which the aromatic to be nitrated is added to the mixture via nozzles and finely distributed. The reaction mixture subsequently flows through the tube reactor R1. This is modularly constructed and consists of pipeline parts of different lengths. According to the invention, between 2 and 20 static mixers are installed between the pipeline parts, and provide the corresponding remixing of the reaction mixture. The reactor R1 is followed by a separator S1, in which the organic phase is separated from the acid phase. The obtained sulfuric acid phase is subsequently passed into a flash evaporator F1 operated in a vacuum. The positioning of the separator S1 relative to F1 is selected in such a way that, in the established vacuum, the acid phase runs from S1 to F1 automatically as a result of the different pressure. Here, water, the organic components still present in the sulfuric acid in accordance with the solubility, and HNO₂ from the sulfuric acid evaporate spontaneously as a result of the flash evaporation, until they are cooled to boiling temperature for the established vacuum. Subsequently, or alternatively in a manner integrated into the flash evaporator, the sulfuric acid is indirectly heated and concentrated to the desired concentration of the re-concentrated sulfuric acid 1 using the concentration unit K1, which in the case of combination with F1 merely consists of a corresponding indirectly heated heat exchanger. In the case of a separate concentrating process following F1, K1 consists of an indirectly heated heat exchanger and an associated evaporator. The part of the re-concentrated sulfuric acid from the flashing and concentrating F1+K1 which is required for adiabatic nitration is fed to the suction side of the pump P1. The water vapours obtained during the evaporation in F1+K1 are condensed (process condensate) either separately or together with the vapours coming from the subsequent sulfuric acid concentration means. Inert gases are removed by a vacuum unit. Preferably, an indirectly cooled vacuum pump is used as a vacuum unit, since both cooling by direct water supply and use of steam-operated vacuum radiators would increase the total wastewater amount.

The remaining sulfuric acid which is not fed to the adiabatic nitration is fed into a subsequent sulfuric acid concentration means, in which the acid is concentrated in one or more steps under vacuum in accordance with the prior art. Here too, an indirectly cooled vacuum pump (or even more than one, depending on performance) is used, since both cooling by direct water supply and the use of steam-operated vacuum radiators would increase the total wastewater amount. If F1+K1 and the sulfuric acid concentration means are operated in the same vacuum, a joint vacuum unit may also be used. All off-gases from the individual process steps are passed to the NOₓ absorption so as to recover any nitrous off-gases present as nitric acid.

For heating and concentrating the acid, the conventional evaporator types, such as natural-circulation evaporators, forced-circulation evaporators, horizontal evaporators, etc., are used. Corresponding processes for sulfuric acid concentration are sufficiently well-known, and are not explained in greater detail here. Corrosion-resistant materials such as enamel or PTFE-lined steel are used as the materials for the reactor R1, the separator S1, the flash evaporator F1, the evaporator at K1 and the evaporator in the sulfuric acid concentration means. For steam-heated heat exchangers such as W2, the heater in K1 and the heaters in the sulfuric acid concentration means, corrosion-resistant materials such as tantalum are used, such as are conventionally used for concentration of sulfuric acid. For acid/acid heat exchangers such as W1, materials such as silicon carbide or tantalum are used. As materials for pipelines in contact with hot sulfuric acid, corrosion-resistant material such as enamel or PTFE-lined steel are used. For apparatuses and machines in contact with process condensate, suitable stainless steels are used.

### BEST MODE OF INVENTION

The following examples further explain the invention.

Example 1 shows by way of example how the individual mass streams can behave in relation to one another. The individual mass streams may of course also vary in ratio and in composition, depending on what process the mass streams come from and how the associated nitration, wash and NOₓ absorption are operated. Example 1 is intended to describe the process according to the invention in greater detail, but the specified values are not intended to limit the process. All % specifications relate to % by weight.

In a nitration process for the production of DNT, 9,859 kg/h waste acid having the following composition is generated: 71% H₂SO₄, 0.8% HNO₃, 1.6% HNO₂, 0.5% DNT, 26.1% H₂O. Further, 2,260 kg/h washing water having the following composition is generated: 8% H₂SO₄, 17% HNO₃, 1% HNO₂, 1% DNT, 73% H₂O. From the NOₓ absorption, 811 kg/h nitric acid comprising 55% HNO₃ and 45% H₂O is recovered. The 3 streams are mixed and preheated to 90° C. Subsequently, 16,086 kg/h uncooled re-concentrated sulfuric acid 1 comprising 75% H₂SO₄ and 25% water is added to the mixture at approximately 112° C., which corresponds to the boiling temperature of the sulfuric acid in the associated re-concentration step at 80 mbar. Into the resulting mixture, which then has a temperature of approximately 102° C., 1,462 kg/h toluene is injected, and the mixture is passed into the adiabatic tube reactor. In the adiabatic tube reactor, the mixture is further mixed by successive static mixers, so as to achieve a complete reaction here. In Example 1, 10 mixing elements are used. At the output of the adiabatic reactor, a mixture is obtained comprising 63.14% H₂SO₄, 0.59% HNO₂, 6.45% MNT, 0.24% DNT, 0.44% toluene and 29.1% H₂O. HNO₃ is neutralised, and is still present only in traces, if at all. The temperature of the reaction mixture has increased to approximately 130° C. as a result of the released reaction energy and the adiabatic reaction regime. The reaction mixture is passed into a separator. At the surface, the organic phase precipitates and is removed. The sulfuric acid phase is then flashed in an evaporator at 80 mbar and pre-concentrated accordingly by the resulting water evaporation. Subsequently, the acid is likewise further concentrated to 75% H₂SO₄ at 80 mbar by indirect heating by the conventional sulfuric acid concentration methods in accordance with the prior art. During the concentration of the sulfuric acid, the dissolved organic compounds and the HNO₂ present are evaporated out of the sulfuric acid together with the water. The vapours are condensed, and the organic phase is separated from the obtained aqueous phase in accordance with the prior art. To prevent DNT deposits, a solvent, in this case MNT, is added to the condensate system in accordance with the prior art. The remaining sulfuric acid which is not fed back to the adiabatic nitration is further concentrated to 93% H₂SO₄ in a vacuum of 80 mbar by indirect steam-heating by the conventional sulfuric acid concentration methods in accordance with the prior art. This re-concentrated sulfuric acid 2 is subsequently cooled, with part of the energy being used as described above for preheating the incoming mixture, and fed to the dinitration.

Example 2 is also intended to show how the individual mass streams can behave with respect to one another. Here, MNT is used instead of toluene as an aromatic for reacting the nitric acid. In a nitration process for producing DNT, 24,951 kg/h waste acid having the following composition is generated: 72% H₂SO₄, 1% HNO₃, 1.3% HNO₂, 0.6% DNT, 25.1% H₂O. 5,650 kg/h washing water having the following composition is further generated: 6% H₂SO₄, 15% HNO₃, 1% HNO₂, 1% DNT, 77% H₂O. From the NOₓ absorption, 1,598 kg/h nitric acid comprising 55% HNO₃ and 45% H₂O is recovered. The 3 streams are mixed and preheated to 85° C. Subsequently, 50,033 kg/h uncooled re-concentrated sulfuric acid 1 comprising 74% H₂SO₄ and 26% water is added to the mixture at approximately 114° C., which corresponds to the boiling temperature of the sulfuric acid in the associated re-concentration step at 100 mbar. Into the resulting mixture, which then has a temperature of approximately 102.6° C., 3,630 kg/h toluene is injected, and the mixture is passed into the adiabatic tube reactor. In the adiabatic tube reactor, the mixture is further mixed by successive static mixers, so as to achieve a complete reaction here. In the example, 15 mixing elements are used. At the output of the adiabatic reactor, a mixture is obtained comprising 63.78% H₂SO₄, 0.44% HNO₂, 0.25% MNT, 6.82% DNT and 28.71% H₂O. HNO₃ is neutralised, and is still present only in traces, if at all. The temperature of the reaction mixture has increased to approximately 125° C. as a result of the released reaction energy and the adiabatic reaction regime. The reaction mixture is passed into a separator. At the surface, the organic phase precipitates and is removed. The sulfuric acid phase is then flashed in an evaporator at 100 mbar and pre-concentrated accordingly by the resulting water evaporation. Subsequently, the acid is likewise further concentrated to 74% H₂SO₄ at 100 mbar by indirect heating, for example steam-heating, by the conventional sulfuric acid concentration methods in accordance with the prior art. The sulfuric acid which is not fed back to the adiabatic nitration is further concentrated to 94% H₂SO₄ in a vacuum of 50 mbar by indirect steam-heating by the conventional sulfuric acid concentration methods in accordance with the prior art.

1. Process for workup of mixed acid and wastewater from the nitration of aromatics, in which the nitric acid present is reacted under adiabatic conditions by reaction with an aromatic, characterised in that
a. at least one waste stream component selected from waste acid (mixed acid) generated in the nitration, acidic washing water from the workup of crude nitroaromatics and dilute nitric acid generated in an off-gas treatment in the course of the nitration is provided,
b. the at least one waste stream component is mixed with re-concentrated sulfuric acid,
c. an aromatic is added to the mixture in stoichiometric excess based on the nitric acid,
d. the obtained reaction mixture is reacted in an adiabatically operated reactor,
e. the obtained organic phase is separated from the sulfuric-acid-containing phase in a separator,
f. the sulfuric-acid-containing phase is concentrated under vacuum and
g. at least one substream of the re-concentrated sulfuric acid from step g) is employed in step b).

2. Process according to claim 1, characterised in that at least two, preferably all, of the aforementioned waste stream components are provided mixed in step a).

3. Process according to either claim 1 or claim 2, characterised in that the at least one waste stream component is preheated in step b) before the re-concentrated sulfuric acid is added.

4. Process according to claim 3, characterised in that the preheating of the mixture is adjusted in such a way that the temperature after the addition of the re-concentrated sulfuric acid is in a range of 70° C. to 130° C., preferably in a range of 90° C. to 110° C.

5. Process according to any of claims 1 to 4, characterised in that the amount and concentration of the re-concentrated sulfuric acid are selected in a manner resulting in a mixture comprising a sulfuric acid content of between 60% and 70% H₂SO₄ and a nitric acid content of between 1% and 5% HNO₃ in step b).

6. Process according to any of claims 1 to 5, characterised in that in step c) the aromatic is added in stoichiometric excess in a range of 1%-20%, preferably 3%-10%, based on the nitric acid amount.

7. Process according to any of claims 1 to 6, characterised in that the operating pressure at the reactor input is in a range of 1 bar to 10 bar, preferably in a range of 3 bar to 6 bar.

8. Process according to any of claims 1 to 7, characterised in that the operating pressure in the separator is between 1 bar and 2 bar.

9. Process according to any of claims 1 to 8, characterised in that the reaction mixture in the reactor is heated by 10 to 40° C. by the adiabatic procedure.

10. Process according to any of claims 1 to 9, characterised in that the sulfuric acid phase is concentrated in a vacuum at a pressure of between 30 mbar and 500 mbar.

11. Process according to any of claims 1 to 10, characterised in that the re-concentrated sulfuric acid which is not used in the process is further concentrated to a final concentration of between 85 and 98% H₂SO₄ in a vacuum of between 150 and 30 mbar, preferably between 100 and 50 mbar.

12. Apparatus for performing the process according to the invention according to any of claims 1 to 11, characterised in that the apparatus consists of a mixing unit, in which the waste acid from the nitration, the recovered dilute nitric acid from the NOₓ absorption and the acidic washing water from the nitroaromatic wash are mixed, at least one heat exchanger for preheating the mixture, a pump for mixing the mixture obtained in the preceding step with sulfuric acid and generating the pressure required at the reactor input, an injection unit in which the aromatic to be nitrated is added to the mixture via nozzles and finely distributed, a modularly constructed tube reactor comprising between 2 and 20 static mixers, a separator in which the organic phase is separated from the acid phase, a flash evaporator, and a concentration unit, comprising an indirectly heated heat exchanger and an associated evaporator having associated vapour condensation and a vacuum unit.

13. Device according to claim 12, characterised in that in addition a single-step or multi-step sulfuric acid concentration means is installed, in which residual sulfuric acid which is not fed to the adiabatic nitration is further concentrated.
